(19) United States
(12) Patent Application Publication
(10) Pub. No.: US 2016/0015987 A1
(43) Pub. Date: Jan. 21, 2016

(54) ULTRA-THIN IMPLANTABLE ENERGY SOURCE

(71) Applicants: COMMISSARIAT A L'ENERGIE ATOMIQUE ET AUX ENERGIES ALTERNATIVES, Paris (FR); UNIVERSITE JOSEPH FOURIER, Grenoble Cedex 9 (FR)

(72) Inventors: Simon PERRAUD, Bandol (FR); Francois BERGER, Meylan (FR); Frederic GAILLARD, Voiron (FR); Nicolas KARST, Folkling (FR); Philippe PANTIGNY, Claix (FR); Emmanuelle ROUVIERE, Le Fontanil Cornillon (FR)

(21) Appl. No.: 14/772,559

(22) PCT Filed: Feb. 27, 2014
(86) PCT No.: PCT/IB2014/059306

§ 371 Date: Sep. 3, 2015

(30) Foreign Application Priority Data
Mar. 8, 2013 (FR) 1352117

(51) Int. Cl. A61N 1/378

(52) U.S. Cl. CPC A61N 1/3787

(57) ABSTRACT

The invention relates to an implantable energy source comprising at least one energy storage sub-system (171) constructed in the form of a stack of thin layers (175) on a substrate (176), characterised in that said energy storage sub-system has a plurality of through-openings (174) for allowing the development and the passage of blood vessels. Preferably, the energy source thereof has a thickness of less than, or equal to, 1 mm, over at least 80% of its surface.

The invention relates to an implantable power source comprising an energy storing subsystem such as a battery, and preferably also an energy harvesting subsystem such as a photovoltaic module allowing said energy storing subsystem to be charged or recharged. Such a power source may serve to power an implantable medical device.

Implantable medical devices (such as cardiac stimulators, cardiac defibrillators, cardiac monitors, neurostimulators, pumps, biomedical sensors, etc.) are generally powered electrically by primary (non-rechargeable) batteries. The primary battery and the medical device to be powered are generally encapsulated in a casing made of titanium or stainless steel, this casing being intended to be implanted under the skin of the patient. The primary battery takes the form of a mechanically rigid and relatively bulky object that occupies a significant proportion of the total volume of the implantable casing. For example, the battery of a cardiac stimulator is typically an object of 5 cm³ volume having a thickness comprised between 0.5 cm and 1 cm and occupying half the volume of the implantable casing. The lifetime of the primary battery, which depends on its energy capacity and the average power consumed by the implantable medical device, is generally comprised between 1 and 10 years. Once discharged, the primary battery must be replaced, thereby implying a surgical operation.

In the last few years, more advantageous novel concepts have been demonstrated in the field of power sources for implantable medical devices. The document “A wireless near-infrared energy system for medical implants” K. Murakawa et al., IEEE Engineering in Medicine and Biology 18, 70, 1999, describes an implantable power source comprising a photovoltaic module and a secondary (rechargeable) battery, the photovoltaic module being electrically connected to the secondary battery in order to allow it to be recharged. This power source is intended to be implanted under the skin of the patient. An external device emitting light in the near infrared is used to carry out a transcutaneous illumination of the implanted photovoltaic module, skin being relatively transparent in the near infrared. The implanted photovoltaic module thus illuminated under infrared generates electrical power allowing the implanted secondary battery to be recharged. In other words, the secondary battery can be recharged by transcutaneous energy transfer, without the need for a surgical operation. This type of implantable power source has the advantage of possessing a longer lifetime than primary batteries. Specifically, the lifetime is no longer limited by the energy capacity of the battery, but rather by the maximum number of charge/discharge cycles the battery can withstand, which may be about several thousand for solid electrolyte batteries.

However, the problem of the bulk and mechanical rigidity of the implantable power source remains to be solved.

Document U.S. Pat. No. 6,961,619 describes an implantable photovoltaic module encapsulated by lamination of polymer sheets, the module and its encapsulation taking the form of an ultra-thin object (a few hundred microns in thickness). This document therefore allows the bulk of the energy harvesting subsystem to be decreased but does not provide a solution to the problem of decreasing the bulk of the complete system, which must not only comprise the energy harvesting subsystem but also the energy storing subsystem and possibly an energy management subsystem and a communication subsystem. In addition, the encapsulation technique provided by the document (lamination of polymer sheets) does not ensure a good hermeticity as polymer materials are known to be poor barriers to moisture and oxygen. This means that there is a risk of the implanted photovoltaic module degrading over time in vivo. Lastly, the implantable photovoltaic module described by the document may take the form of an object of relatively large area (of several centimeters squared or even several tens of centimeters squared), thereby possibly leading to a problem with poor vascularization of the biological tissues of the patient and resulting in necrosis of these tissues.

The article by N. J. Dudney “Thin Film Micro-Batteries”, The Electrochemical Society Interface, autumn 2008, pages 44-48, describes thin-film electrochemical batteries having a thickness of only a few tens of microns, whereas document US 2002/0092558 describes an ultra-thin device integrating thin-film photovoltaic cells and thin-film batteries. These documents do not relate to implantable devices.

The invention aims to solve the problems of the aforementioned prior art, and in particular that of poor vascularization of the biological tissues in which the ultra-thin implantable power source having a relatively large area is implanted. More generally, the invention aims to ensure a better compatibility of the implantable power source with the host organism, whether human or animal.

According to the invention, these aims are achieved with an implantable power source comprising at least one energy storing subsystem produced in the form of a thin-film stack on a substrate, characterized in that said energy storing subsystem has a plurality of through-apertures in order to allow the development and passage of blood vessels.

Advantageously, each said aperture may have an area comprised between 0.01 mm² and 4 mm². Furthermore, the spacing between said apertures may advantageously be comprised between 1 mm and 1 cm.

Such an implantable power source may have a biocompatible coating covering at least one portion of its surface comprising the interior surface of said apertures. In particular, the power source may comprise an exterior film made of a biocompatible organic material and an interior film made of an inorganic material that is impermeable to moisture and oxygen. Said biocompatible coating may be substantially transparent at least in a spectral range in the visible or near infrared; this is important if, as will be discussed below, the power source incorporates a photovoltaic module.

In order to facilitate the development of blood vessels through the power source, said apertures may be completely or partially filled with a gel promoting cellular growth.

Said energy storing subsystem may have a plurality of active regions separated by interconnect regions, at least certain of said apertures being produced in said active regions and/or in said interconnect regions.

The implantable power source may also comprise at least one energy harvesting subsystem connected to said energy storing subsystem so as to allow the latter to be charged, said energy harvesting subsystem being in turn produced in the form of a thin-film stack on a substrate and having a plurality of said through-apertures. In particular, said energy harvesting subsystem may be chosen from a thin-film photovoltaic module and a thin-film spiral coil. As a variant, said energy harvesting subsystem may for example be a piezoelectric generator, producing electrical power from movements of the host organism, or a thermoelectric generator, producing electrical power from temperature gradients inside the host organism.

Just like the energy storing subsystem, said energy harvesting subsystem may have at least one active region and at least one inactive or interconnect region, at least certain of said apertures being produced in said active region(s) and/or in said inactive or interconnect region(s).

According to Various Embodiments:
Said energy storing subsystem and said energy harvesting subsystem may comprise thin-film stacks deposited on or transferred to respective substrates and be in turn stacked to form a power source of unitary construction.
Said energy storing subsystem and said energy harvesting subsystem may be stacked on a common substrate to form a power source of unitary construction.
Said energy storing subsystem and said energy harvesting subsystem may comprise thin-film stacks deposited on or transferred to two opposite sides of a common substrate to form a power source of unitary construction.

As a variant, said energy storing subsystem and said energy harvesting subsystem may be arranged side-by-side, in which case the power source may not be of unitary construction.

Advantageously, said or each said substrate may be flexible or shapeable in order to more easily adapt to the host organism and engender less discomfort and fewer internal lesions.

Advantageously, such an implantable power source may have over the entirety of its area, or over at least 80% of the latter if it also comprises subsystems that are difficult to produce in thin-film technologies, a thickness smaller than or equal to 1 mm (and therefore “ultra-thin”).

Another subject of the invention is an implantable device comprising an implantable power source as claimed in one of the preceding claims and a medical apparatus connected to said energy storing subsystem in order to be powered. Advantageously, said medical device may in turn be ultra-thin.

Other features, details and advantages of the invention will become apparent on reading the description given with reference to the appended figures, which are given by way of example and show, respectively:

FIG. 1, a cross-sectional view of an implantable power source comprising a thin-film photovoltaic module;

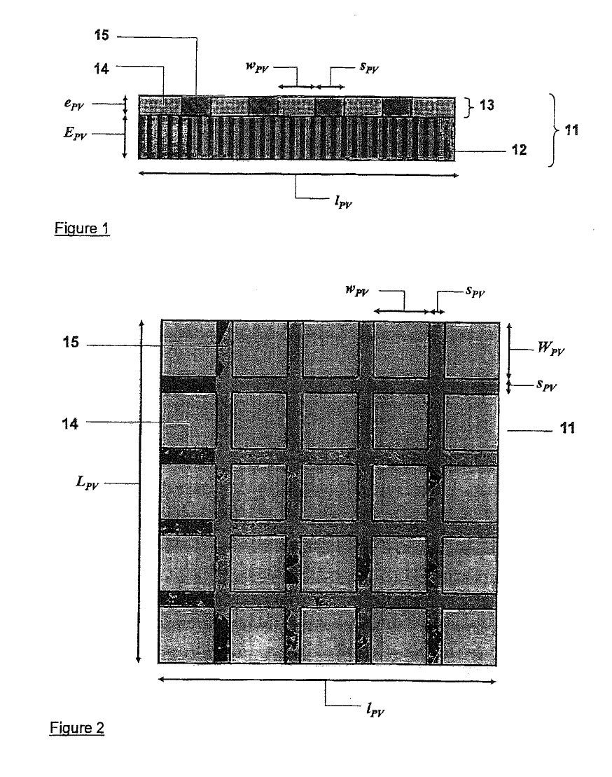

FIG. 2, a top view of the same power source;

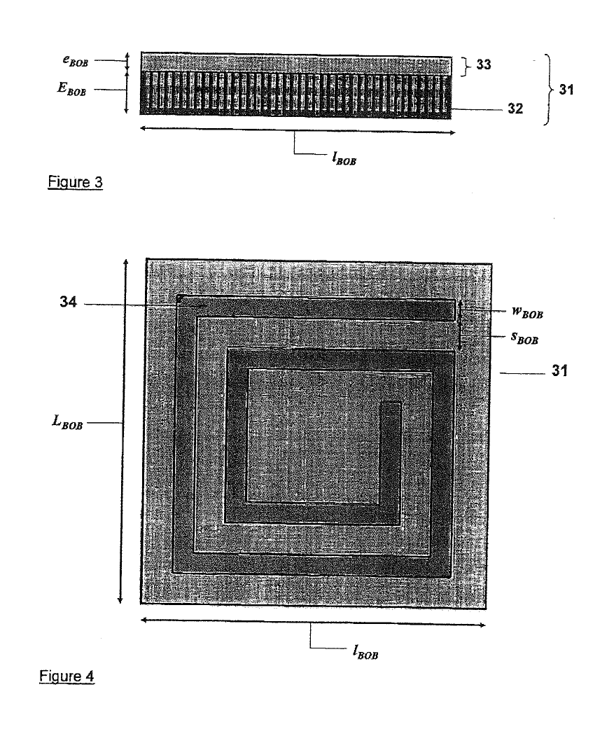

FIG. 3, a cross-sectional view of an implantable power source comprising a thin-film spiral coil;

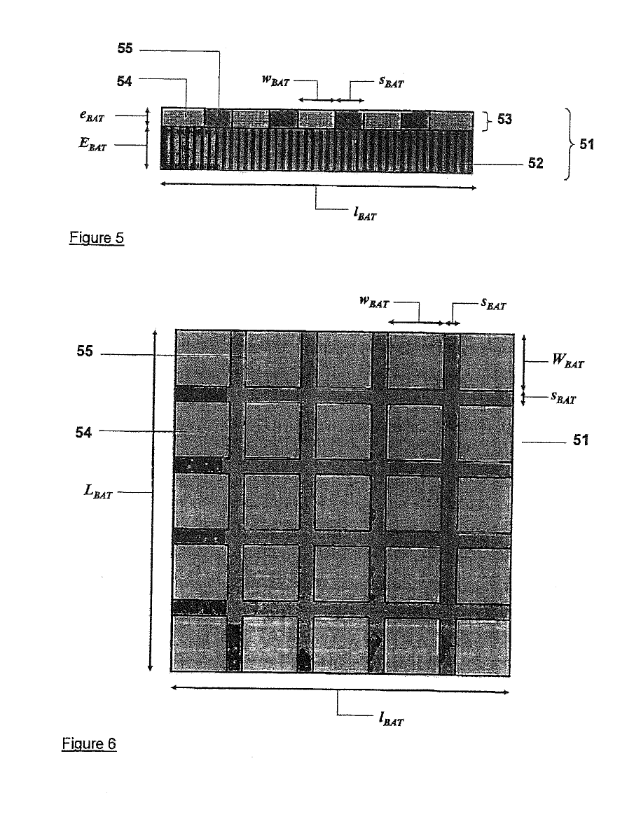

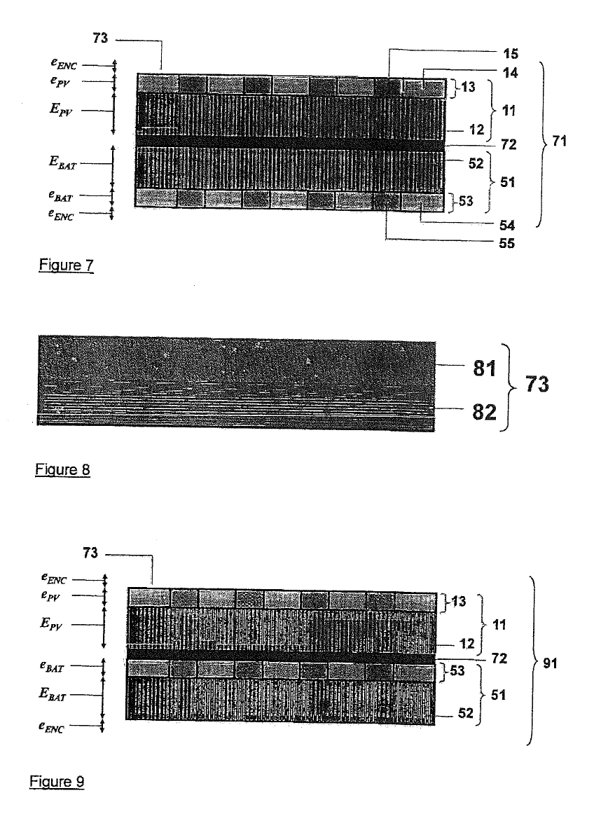

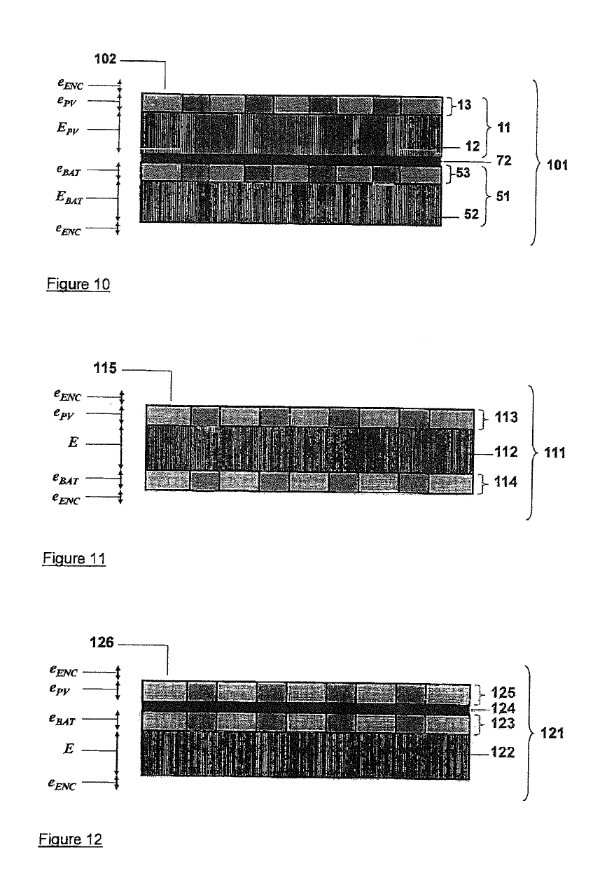

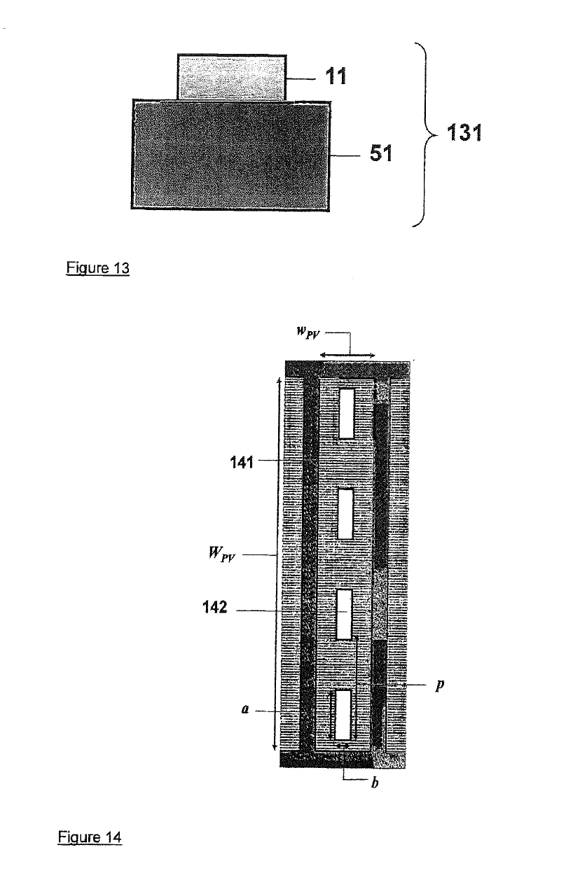

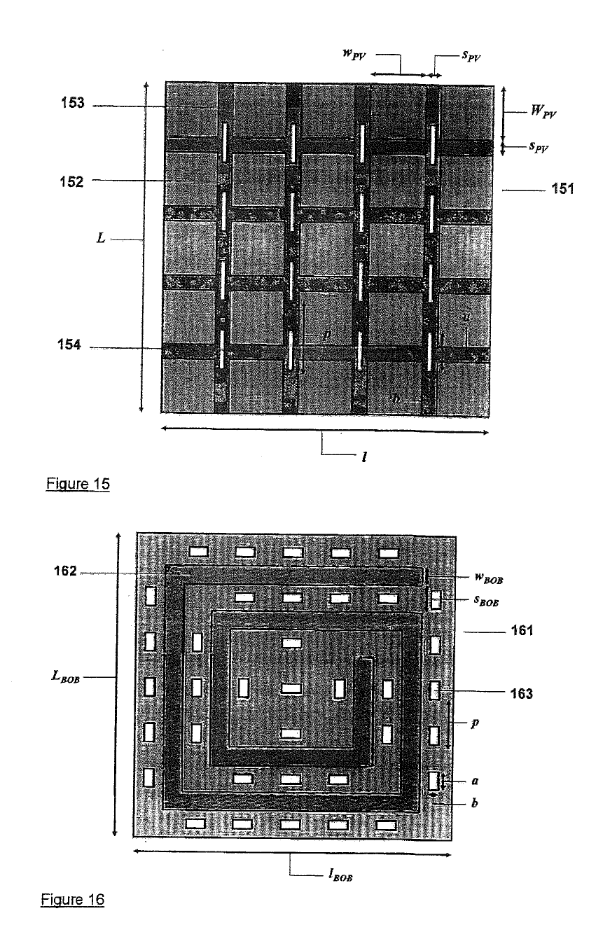

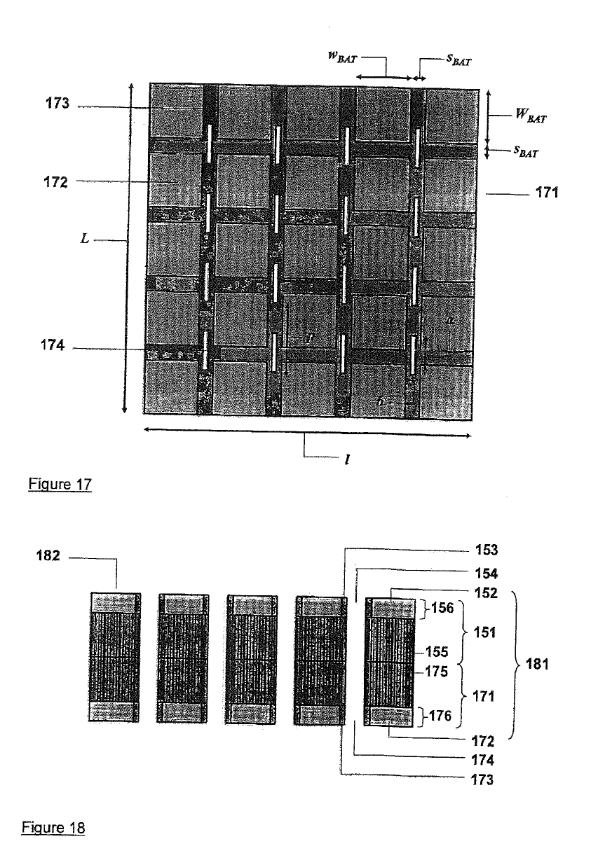

FIG. 4, a top view of the same power source;

FIG. 5, a cross-sectional view of an implantable power source comprising a thin-film electrical battery;

FIG. 6, a top view of the same power source;

FIG. 7, a cross-sectional view of an implantable power source comprising a thin-film photovoltaic module and a thin-film battery;

FIG. 8, a cross-sectional view of a thin-film stack forming a coating allowing an implantable power source to be encapsulated;

FIGS. 9 to 13, various variants of an implantable power source comprising a thin-film photovoltaic module and a thin-film battery;

FIGS. 14 and 15, various ways of arranging through-apertures in a photovoltaic module of an implantable power source;

FIG. 16, one way of arranging through-apertures in a spiral coil of an implantable power source;

FIG. 17, one way of arranging through-apertures in a battery of an implantable power source; and

FIG. 18, a cross-sectional view of an implantable power source comprising a thin-film photovoltaic module and a thin-film battery, having through-apertures (or through-holes).

An active implantable device such as a cardiac stimulator, defibrillator and/or monitor, a neurostimulator, a pump, a sensor, etc., necessarily comprises a power source that is also implantable. Such a power source comprises at least one subsystem for storing electrical energy—generally a battery—powering a medical apparatus, and preferably also an energy harvesting subsystem allowing the storing subsystem to be recharged.

The energy harvesting subsystem of the rechargeable implantable power source may comprise a thin-film photovoltaic module, such as the photovoltaic module 11 illustrated in FIG. 1 (cross-sectional view) and in FIG. 2 (top view). The expression “thin-film” is understood to mean made up of films of thickness smaller than or equal to 500 μm, preferably 200 μm and even more preferably 100 μm. In this case, the rechargeable implantable power source may be recharged by transcutaneous transfer of light energy, preferably with light in the near infrared (i.e. in the spectral range extending from 700 to 1200 nm). The photovoltaic module 11 is rectangular in shape, of length denoted L_{PV} and of width denoted I_{PV}. Advantageously, the length L_{PV} and width I_{PV} are comprised between 100 μm and 10 cm, i.e. the area of the photovoltaic module 11 is comprised between 0.01 mm² and 100 cm². Preferably, the length L_{PV} and width I_{PV} are comprised between 1 and 5 cm, i.e. the area of the photovoltaic module 11 is comprised between 1 and 25 cm². The photovoltaic module 11 is formed from a substrate 12 onto which a thin-film stack 13 has been deposited or transferred. The substrate 12 is for example a silicon wafer, a glass wafer, a sheet of metal (stainless steel, titanium, etc.) or a sheet of polymer (polyethylene, polytetrafluoroethylene, polyimide, polyester, etc.). Advantageously, the substrate 12 is a substrate that is mechanically flexible or shapeable, such as a sheet of metal or a sheet of polymer. Advantageously, the thickness of the substrate 12, denoted E_{PV}, is smaller than 200 μm and preferably smaller than 50 μm. The thin-film stack 13 comprises materials for forming electrodes (metals, transparent conductive oxides, etc.) and light absorber materials [single-crystal or polycrystalline silicon (Si), single-crystal indium phosphide (InP), amorphous silicon-germanium (a-SiGe), microcrystalline silicon (μc-Si), Cu(In, Ga)(Se, S)₂ (CIGS), Cu₂ZnSn(Se, S)₄ (CZTS), etc.]. The thin-film stack 13 may also comprise materials for forming electrical interconnects (metals). The thin-film stack 13 may also comprise films playing only a mechanical role (for example a role as a carrier or a role in adhesion). The thin films of the stack 13 may be deposited on the substrate 12 using a deposition technique suitable for producing thin films (physical vapor deposition, chemical vapor deposition, electrodeposition, deposition by coating or printing, etc.). The thin films of the stack 13 may also be transferred to the substrate 12 using transfer techniques suitable for producing thin films (for example by way of a laminating process using an adhesive material). The thin films of the stack 13 may be structured, during their deposition or transfer step (for example by deposition of the thin films through a stencil or by deposition of the thin films by printing), or after they have been deposited or transferred (for example by photolithography, chemical etching, plasma etching, mechanical etching or laser etching). Advantageously, the maximum thickness of the thin-film stack 13, denoted e_{PV}, is smaller than 50 μm and preferably smaller than 5 μm. The thin-film stack 13 is subdivided into active zones, such as the zone 14, and interconnect zones, such as the zone 15. The active zones are rectangular in shape, of length denoted W_{PV} and of width denoted w_{PV}. Advantageously, the length W_{PV} and the width w_{PV} are comprised between 100 μm and 10 cm, i.e. the area of an active zone is comprised between 0.01 mm² and 100 cm². Preferably, the length W_{PV} and the width w_{PV} are larger than 1 mm, i.e. the area of an active zone is larger than 1 mm². The interconnect zones take the form of rectangular strips of width, denoted s_{PV}, advantageously smaller than 1 cm and preferably smaller than 1 mm. Only the active zones contribute to the generation of electrical power. Each active zone corresponds to a photovoltaic cell. The interconnect zones allow two photovoltaic cells defined on the same substrate 12 to be electrically interconnected. The interconnection may be a monolithic interconnection (i.e. obtained by structuring the thin films with a set of 3 trenches, generally designated P1, P2 and P3, as described for example in the document “Transparent electrode requirements for thin film solar cell modules”, M. W. Rowell et al., Energy Environ. Sci. 4, 131, 2010). In this case, it is a question of a series interconnection. Interconnection may also be achieved using metal tracks or wires. In this case, it may be a question of a series or parallel interconnection. The photovoltaic module 11 therefore consists of an array of photovoltaic cells interconnected in series or in parallel. The voltage and the current of the photovoltaic module 11 depend on the number of photovoltaic cells that the module comprises and on the (series or parallel) interconnection schema. Interconnection in series promotes high voltages and low currents whereas interconnection in parallel promotes low voltages and high currents.

One particularly advantageous process for fabricating the photovoltaic module 11 comprises the following steps:

1) depositing on the substrate 12 a back electrode by physical or chemical vapor deposition;

2) structuring the back electrode by laser etching, in order to define the P1 monolithic interconnection trenches;

3) depositing an absorber by physical or chemical vapor deposition;

4) structuring the absorber by mechanical or laser etching, in order to define the P2 monolithic interconnection trenches;

5) depositing a front electrode by physical or chemical vapor deposition; and

6) structuring the front electrode by mechanical or laser etching, in order to define the P3 monolithic interconnection trenches.

By way of example, let us consider a thin-film photovoltaic cell implanted under the skin of a patient, and that is illuminated in the near infrared (i.e. in the spectral range extending from 750 to 1200 nm) with a power density of about a few mW/cm² or a few tens of mW/cm² (typical infrared power density that may be transmitted through human skin without risk of burns). Under these conditions, the photovoltaic cell generally produces a voltage at the maximum power point of about a few hundred mV, and a current at the maximum power point of about a few mA/cm² or a few tens of mA/cm². More precisely, let us consider an implanted photovoltaic cell based on thin films of CIGS, and that is illuminated in the near infrared at a wavelength of 850 nm and with a power density of 2 mW/cm². Under these conditions, the CIGS photovoltaic cell may produce a voltage at the maximum power point of about 400 mV and a current at the maximum power point of about 1.5 mA/cm², i.e. a maximum electrical power of about 0.6 mW/cm². Let us consider a photovoltaic module 11 based on thin films of CIGS, having the following dimensions: L_{PV}=50 mm, I_{PV}=53 mm, W_{PV}=L_{PV}=50 mm, w_{PV}=8 mm, and s_{PV}=1 mm. In this case, the photovoltaic module 11 comprises 6 CIGS photovoltaic cells, each cell having an active area of 4 cm². Let us consider a series interconnection schema. Once implanted, the photovoltaic module 11 may therefore produce a voltage at the maximum power point of about 2.4 V and a current at the maximum power point of about 6 mA, i.e. a maximum electrical power of about 14 mW.

The photovoltaic module is not necessarily rectangular in shape. In particular, the photovoltaic module may have rounded edges. The active zones are not necessarily rectangular in shape. The interconnect zones do not necessarily take the form of rectangular strips.

The energy harvesting subsystem of the rechargeable implantable power source may comprise an array of thin-film photovoltaic modules. Each photovoltaic module consists of a substrate onto which a thin-film stack has been deposited or transferred. The various photovoltaic modules may be positioned in the same plane. The various photovoltaic modules may be electrically interconnected in series or in parallel. This allows the voltage or the current of the energy harvesting subsystem to be increased.

The energy harvesting subsystem of the rechargeable implantable power source may comprise a thin-film spiral coil, such as the coil 31 illustrated in FIG. 3 (cross-sectional view) and in FIG. 4 (top view). In this case, the rechargeable implantable power source may be recharged by transcutaneous transfer of energy by inductive coupling. The coil 31 is rectangular in shape, of length denoted L_{BOB} and of width denoted I_{BOB}. Advantageously, the length L_{BOB} and width I_{BOB} are comprised between 100 μm and 10 cm, i.e. the area of the coil 31 is comprised between 0.01 mm² and 100 cm². Preferably, the length L_{BOB} and width I_{BOB} are comprised between 1 and 5 cm, i.e. the area of the coil 31 is comprised between 1 and 25 cm². The coil 31 is formed from a substrate 32 onto which a thin-film stack 33 has been deposited or transferred. The substrate 32 is for example a glass wafer or a sheet of polymer (polyethylene, polytetrafluoroethylene, polyimide, polyester, etc.). Advantageously, the substrate 32 is a substrate that is mechanically flexible or shapeable, such as a sheet of polymer. Advantageously, the thickness of the substrate 32, denoted E_{BOB}, is smaller than 200 μm and preferably smaller than 50 μm. The thin films of the stack 33 may be deposited on the substrate 32 using a deposition technique suitable for producing thin films (physical vapor deposition, chemical vapor deposition, electrodeposition, deposition by coating or printing, etc.). The thin films of the stack 33 may also be transferred to the substrate 32 using transfer techniques suitable for producing thin films (for example by way of a laminating process using an adhesive material). The thin films of the stack 33 may be structured, during their deposition or transfer step (for example by deposition of the thin films through a stencil or by deposition of the thin films by printing), or after they have been deposited or transferred (for example by photolithography, chemical etching, plasma etching, mechanical etching or laser etching). Advantageously, the maximum thickness of the thin-film stack 33, denoted e_{BOB}, is smaller than 100 μm and preferably smaller than 50 μm. The thin-film stack 33 comprises a spiral-shaped metal track 34. The width of the segments of the track 34 is denoted w_{BOB} and the spacing between the segments of the track 34 is denoted s_{BOB}. Advantageously, the distances w_{BOB} and s_{BOB} are comprised between a few μm and a few mm.

The spiral coil is not necessarily rectangular in shape. In particular, the spiral coil may have rounded edges.

The energy storing subsystem of the rechargeable implantable power source may comprise a thin-film secondary battery, such as the secondary battery 51 illustrated in FIG. 5 (cross-sectional view) and FIG. 6 (top view). The secondary battery 51 is rectangular in shape, of length denoted L_{BAT} and of width denoted I_{BAT}. Advantageously, the length L_{BAT} and width I_{BAT} are comprised between 100 μm and 10 cm, i.e. the area of the secondary battery 51 is comprised between 0.01 mm² and 100 cm². Preferably, the length L_{BAT} and width I_{BAT} are comprised between 1 and 5 cm, i.e. the area of the secondary battery 51 is comprised between 1 and 25 cm². The secondary battery 51 is formed from a substrate 52 onto which a thin-film stack 53 has been deposited or transferred. The substrate 52 is for example a silicon wafer, a glass wafer, a sheet of metal (stainless steel, titanium, etc.) or a sheet of polymer (polyethylene, polytetrafluoroethylene, polyimide, polyester, etc.). Advantageously, the substrate 52 is a substrate that is mechanically flexible or shapeable, such as a sheet of metal or a sheet of polymer. Advantageously, the thickness of the substrate 52, denoted E_{EAT}, is smaller than 200 μm and preferably smaller than 50 μm. The thin-film stack 53 comprises current collector materials (metals), materials for forming cathodes (TiOₓS_{y}, LiCoO₂, LiMn₂O₄, LiFePO₄, V₂O₅, etc.), materials for forming electrolytes (solid electrolytes such as Lipon, which is a material based on lithium, phosphorus, oxygen and nitrogen), and materials for forming anodes (Li, or an alloy of Li with C, Si, Ge and/or Sn). The thin-film stack 53 may also comprise materials for forming electrical interconnects (metals). The thin-film stack 53 may also comprise films playing only a mechanical role (for example a role as a carrier or a role in adhesion). The thin films of the stack 53 may be deposited on the substrate 52 using a deposition technique suitable for producing thin films (physical vapor deposition, chemical vapor deposition, electrodeposition, deposition by coating or printing, etc.). The thin films of the stack 53 may also be transferred to the substrate 52 using transfer techniques suitable for producing thin films (for example by way of a laminating process using an adhesive material). The thin films of the stack 53 may be structured, during their deposition or transfer step (for example by deposition of the thin films through a stencil or by deposition of the thin films by printing), or after they have been deposited or transferred (for example by photolithography, chemical etching, plasma etching, mechanical etching or laser etching). Advantageously, the maximum thickness of the thin-film stack 53, denoted e_{BAT}, is smaller than 50 μm and preferably smaller than 20 μm. The thin-film stack 53 is subdivided into active zones, such as the zone 54, and interconnect zones, such as the zone 55. The active zones are rectangular in shape, of length denoted W_{BAT} and of width denoted w_{BAT}. Advantageously, the length W_{BAT} and the width w_{BAT} are comprised between 100 μm and 10 cm, i.e. the area of an active zone is comprised between 0.01 mm² and 100 cm². Preferably, the length W_{BAT} and the width w_{BAT} are larger than 1 mm, i.e. the area of an active zone is larger than 1 mm². The interconnect zones take the form of rectangular strips of width, denoted s_{BAT}, advantageously smaller than 1 cm and preferably smaller than 1 mm. Only the active zones contribute to the storage of electrical energy. Each active zone corresponds to a electrochemical cell. The interconnect zones allow two electrochemical cells defined on the same substrate 52 to be electrically interconnected. Electrical interconnection may be achieved using metal tracks or wires. It may be a question of a series or parallel interconnection. The secondary battery 51 therefore consists of an array of electrochemical cells interconnected in series or in parallel. The voltage and the capacity of the secondary battery 51 depend on the number of electrochemical cells that the secondary battery comprises (this number is higher than or equal to 1) and on the (series or parallel) interconnection schema. Interconnection in series promotes high voltages and low capacities whereas interconnection in parallel promotes low voltages and high capacities.

One particularly advantageous process for fabricating the secondary battery 51 comprises the following steps:

1) transferring to the substrate 52 a metal film by lamination using an adhesive material;

2) structuring the metal film by chemical etching, in order to define electrical interconnects;

3) fabricating a plurality of electrochemical cells, each electrochemical cell consisting of a polymer sheet on which have been deposited in succession a back current collector, a cathode, a solid electrolyte, an anode, and a front current collector;

4) testing separately each electrochemical cell, in order to determine which cells are functional cells (i.e. cells meeting specification in terms of electrical performance); and

5) transferring the functional electrochemical cells to the substrate 52 by lamination using an adhesive material, and electrically connecting these cells to the interconnect zones defined in step 2).

Such a process is advantageous as the manufacturing yield of electrochemical cells (i.e. the fraction of functional cells) may be relatively low, typically lower than 80%.

A thin-film electrochemical cell generally has a voltage of a few volts and a capacity of a few hundred μA.h/cm², i.e. an energy capacity possibly of about a few mW.h/cm². More precisely, an electrochemical cell based on a TiOₓS_{y}/Lipon/Li thin-film stack may have a voltage of about 2.5 V and a capacity of about 300 μA.h/cm², i.e. an energy capacity of about 750 μW.h/cm². Let us consider a secondary battery 51 based on a TiOₓS_{y}/Lipon/Li thin-film stack having the following dimensions: L_{BAT}=50 mm, I_{BAT}=44 mm, W_{BAT} 4.1 mm, w_{BAT}=8 mm, and s_{BAT}=1 mm. In this case, the secondary battery 51 comprises 50 electrochemical cells, each electrochemical cell having an active area of 0.328 cm². Let us consider a parallel interconnection schema.

The secondary battery 51 may therefore have a voltage of about 2.5 V and a capacity of about 4.9 mA.h, i.e. an energy capacity of about 12 mW.h.

The secondary battery is not necessarily rectangular in shape. In particular, the secondary battery may have rounded edges. The active zones are not necessarily rectangular in shape. The interconnect zones do not necessarily take the form of rectangular strips.

The energy storing subsystem of the rechargeable implantable power source may comprise a bank of thin-film secondary batteries. Each secondary battery consists of a substrate onto which a thin-film stack has been deposited or transferred. The various secondary batteries may be stacked on top of each other, or positioned in the same plane. The various secondary batteries may be electrically interconnected in series or in parallel. This allows the voltage or the capacity of the energy storing subsystem to be increased.

A rechargeable implantable power source 71 may comprise an assembly of the thin-film photovoltaic module 11 and the thin-film secondary battery 51, as illustrated in FIG. 7 (cross-sectional view). The photovoltaic module 11 is electrically connected to the secondary battery 51 in order to allow the secondary battery 51 to be recharged. In the case illustrated in FIG. 7, the photovoltaic module 11 is positioned above the secondary battery 51. The dimensions of the secondary battery 51 are advantageously equal to the dimensions of the photovoltaic module 11, i.e. L_{BAT}=L_{PV} and I_{BAT}=I_{PV}. In the case illustrated in FIG. 7, the photovoltaic module 11 and the secondary battery 51 are assembled back-to-back, the back side of the substrate 12 (i.e. the side not holding the thin-film stack 13) being fastened to the back side of the substrate 52 (i.e. the side not holding the thin-film stack 53). The substrates may be fastened together by lamination using an adhesive polymer sheet 72 (polyimide, polyester, polyacrylic, etc.). The photovoltaic module 11 and the secondary battery 51 may be electrically interconnected using techniques known in the field of printed circuit boards, for example by vertical interconnect accesses (vias). The assembly formed by the photovoltaic module 11 and the secondary battery 51 is encapsulated with a film stack 73. Advantageously, the maximum thickness of the stack 73, denoted e_{ENC}, is smaller than 200 μm (preferably smaller than 50 μm). The maximum thickness of the rechargeable implantable power source 71 is about equal to E_{PV}+e_{PV}+E_{BAT}+e_{BAT}+2×e_{ENC}. Advantageously, the maximum thickness of the rechargeable implantable power source 71 is smaller than 1 mm and preferably smaller than 300 μm.

By way of example, let us consider a rechargeable implantable power source 71 comprising a photovoltaic module 11 based on thin films of CIGS, and a secondary battery 51 based on a TiOₓS_{y}/Lipon/Li thin-film stack. The photovoltaic module 11 is electrically connected to the secondary battery 51 in order to allow the secondary battery 51 to be recharged. We saw above that the photovoltaic module 51, when illuminated in the near infrared at a wavelength of 850 nm and with a power density of 2 mW/cm², may produce a voltage at the maximum power point of about 2.4 V and a current at the maximum power point of about 6 mA, i.e. a maximum electrical power of about 14 mW. We also saw above that the secondary battery may have a voltage of about 2.5 V and a capacity of about 4.9 mA.h, i.e. an energy capacity of about 12 mW.h. The photovoltaic module is therefore capable of completely recharging the secondary battery in about 50 minutes. In other words, the rechargeable implantable power source may be completely recharged in about 50 minutes. If the rechargeable implantable power source powers an implantable medical device such as a cardiac stimulator consuming an average electrical power typically of 30 μW, then the autonomy of the rechargeable implantable power source (i.e. the maximum length of time between two recharges) is about 16 days. A longer autonomy may be obtained using a stack of a plurality of thin-film secondary batteries.

The film stack 73 ensuring the encapsulation comprises at least two films, as illustrated in FIG. 8 (cross-sectional view). The external film 81, i.e. the film intended to make contact with biological tissues, is formed from a biocompatible material that may be a biocompatible polymer material (parylene, polysiloxanes, etc.) or a biocompatible inorganic material (alumina, zirconia, etc.). Apart from its biocompatibility, the advantage of parylene, polysiloxanes, alumina and zirconia is that they have a good transparency to radiation in the near infrared. However, parylene and polysiloxanes, like most polymer materials, are poor barriers to moisture and oxygen. A single thin film of alumina or zirconia is also not an excellent barrier to moisture and oxygen, because of the presence of defects. The internal films 82, i.e. the films that are not intended to make contact with biological tissues, increase the barrier to moisture and oxygen. Advantageously, the internal films 82 consist of a film stack made up of organic films (for example based on acrylates) in alternation with non-metal inorganic films (for example based on alumina, silicon oxide, silicon nitride or silicon carbide) or with extremely thin metal films (thickness smaller than 10 nm). Apart from the fact of forming an effective barrier to moisture and oxygen, the advantage of this type of stack is that it has a good transparency to radiation in the near infrared. The internal films 82 are not necessarily biocompatible, thereby ensuring a vast choice of materials or combinations of materials.

The process allowing the assembly formed by the photovoltaic module 11 and the secondary battery 51 to be encapsulated may be a lamination process or a process for depositing thin films, or a combination of these two types of process. Among processes for depositing thin films, chemical vapor deposition and atomic layer deposition (ALD) processes are preferred because they allow conformal thin films to be obtained, i.e. thin films the thickness of which is substantially constant whatever the spatial orientation of the surface on which they are deposited (for example, the thickness deposited on a horizontal surface is similar to that deposited on a vertical surface).

The way in which the photovoltaic module 11 is assembled with the secondary battery 51 may be different from that illustrated in FIG. 7. For example, in the case of the rechargeable implantable power source 91 illustrated in FIG. 9 (cross-sectional view), the back side of the substrate 12 is fastened to the front side of the substrate 52 (i.e. the side holding the thin-film stack 53). Relative to the rechargeable implantable power source 71 illustrated in FIG. 7, the rechargeable implantable power source 91 has the following advantage: the substrate 52 (in particular if it is a question of a biocompatible metal substrate such as a substrate made of titanium or stainless steel, or of a polymer substrate the back side of which is covered with a biocompatible metal film) may play the role of a biocompatible film and act as a barrier to moisture and oxygen, ensuring excellent protection of the thin-film stack 53. Therefore, it is not absolutely necessary for the film stack 73 to extend over the back side of the substrate 52, thereby making the encapsulation process easier to carry out. Thus, in the case of the rechargeable implantable power source 101 illustrated in FIG. 10 (cross-sectional view), the film stack 102 ensuring the encapsulation does not cover the back side of the substrate 52. It will also be noted that in this configuration the substrate 12 and the thin-film stack 13 may play the role of barrier films to moisture and oxygen, thus increasing the protection of the thin-film stack 53. Generally, the thin-film stack 53 of the secondary battery is more sensitive to moisture and oxygen than the thin-film stack 13 of the photovoltaic module, in particular because of the presence of lithium.

Alternatively, a rechargeable implantable power source 111 may comprise a thin-film photovoltaic module and a thin-film secondary battery that are fabricated on two opposite sides of the same substrate 112 of thickness denoted E as illustrated in FIG. 11 (cross-sectional view). A thin-film stack 113 of maximum thickness denoted e_{PV} has been deposited on or transferred to one side of the substrate 112 in order to form the photovoltaic module, and a thin-film stack 114 of maximum thickness denoted e_{BAT} has been deposited on or transferred to the other side of the substrate 112 in order to form the secondary battery. The assembly formed by the photovoltaic module and the secondary battery is encapsulated with a film stack 115, of maximum thickness denoted e_{ENC}. Relative to the rechargeable implantable power sources 71, 91 and 101 illustrated in FIGS. 7, 9 and 10, the rechargeable implantable power source 111 has the advantage of being able to be thinner, because the thickness of one substrate is saved. Thus, the maximum thickness of the rechargeable implantable power source 111 is about equal to E+e_{PV}+e_{BAT}+2×e_{ENC}.

Alternatively, a rechargeable implantable power source 121 may comprise a thin-film photovoltaic module and a thin-film secondary battery that are fabricated on the same side of a given substrate 122 of thickness denoted E as illustrated in FIG. 12 (cross-sectional view). A thin-film stack 123 of maximum thickness denoted e_{BAT} has been deposited on or transferred to the substrate 122 in order to form the secondary battery, then a planarization film 124 has been deposited on or transferred to the stack 123, then a thin-film stack 125 of maximum thickness denoted e_{PV} has been deposited on or transferred to the film 124 in order to form the photovoltaic module. The assembly formed by the photovoltaic module and the secondary battery is encapsulated with a film stack 126, of maximum thickness denoted e_{ENC}. Relative to the rechargeable implantable power sources 71, 91 and 101 illustrated in FIGS. 7, 9 and 10, the rechargeable implantable power source 121 has the advantage of being able to be thinner, because the thickness of one substrate is saved. Thus, the maximum thickness of the rechargeable implantable power source 121 is about equal to E+e_{PV}+e_{BAT}+2×e_{ENC}. Relative to the rechargeable implantable power source 111 illustrated in FIG. 11, the rechargeable implantable power source 121 has the following advantage: the substrate 122 (in particular if it is a question of a biocompatible metal substrate such as a substrate made of titanium or stainless steel, or of a polymer substrate the back side of which is covered with a biocompatible metal film) may play the role of a biocompatible film and act as a barrier to moisture and oxygen, ensuring excellent protection of the thin-film stack 123. Therefore, it is not absolutely necessary for the film stack 126 to extend over the back side of the substrate 122, thereby making the encapsulation process easier to carry out. It will also be noted that in this configuration the thin-film stack 125 may play the role of a barrier film to moisture and oxygen, thus increasing the protection of the thin-film stack 123.

The photovoltaic module 11 is not necessarily positioned above the secondary battery 51, but may also be positioned in the same plane as the secondary battery 51, as in the case of the rechargeable implantable power source 131 illustrated in FIG. 13 (top view). The dimensions of the secondary battery 51 are not necessarily equal to those of the photovoltaic module 11.

The rechargeable implantable power sources 71, 91, 101, 111, 121 and 131 take the form of ultra-thin objects that are advantageously mechanically flexible or shapeable. Thus, the rechargeable implantable power sources 71, 91, 101, 111, 121 and 131 may be implanted in zones accessible to a conventional bulky and mechanically rigid power source, but with the advantage of considerably improving the comfort of the patient, for example:
under the skin of the chest, in order to power a cardiac stimulator, a cardiac sensor, a neuronal stimulator, or a vagal stimulator; or
under the skin of the abdomen, to power a medullary stimulator or a pump.

The rechargeable implantable power sources 71, 91, 101, 111, 121 and 131 may also be implanted in zones of the human body that are not easily accessible to a conventional bulky and mechanically rigid power source. For example, the rechargeable implantable power sources 71, 91, 101, 111, 121 and 131 may be implanted:
under the skin of the head, in order to power a neuronal stimulator;
under the skin of the neck or nape, in order to power a vagal stimulator; or
partially or completely wound around the vagus nerve in the neck, in order to power a vagal stimulator.

In these examples, the system to be powered (electrical stimulation system) may be integrated with the rechargeable implantable power source, into a single and only ultra-thin object that is advantageously mechanically flexible or shapeable. In these examples, it is important to note that the rechargeable implantable power source is implanted in proximity to the zone to be electrically stimulated (brain in the case of neuronal stimulation, vagus nerve in the case of vagal stimulation). In contrast, a conventional bulky and mechanically rigid power source can be implanted only relatively far from the zone to be electrically stimulated (typically the conventional power source of a neuronal stimulator or of a vagal stimulator is implanted in the chest). Therefore, the use of rechargeable implantable power sources according to the invention allows the length of the probes transporting the therapeutic electrical pulses to be considerably decreased, this having many advantages, for example in terms of surgical operations, the comfort of the patient, and compatibility with magnetic resonance imaging (MRI).

The rechargeable implantable power source 131 may be implanted under the skin of the head in order to power a neuronal stimulator, the photovoltaic module 11 being positioned under the skin of the forehead and the secondary battery 51 being partially or completely positioned under the scalp. Positioning the rechargeable implantable power source 131 in this way has the following advantages:
the photovoltaic module 11 may receive a high density of luminous power, because the photovoltaic module 11 is positioned in a zone without hair. This makes it possible to ensure a rapid recharge of the rechargeable implantable power source 131;
the thickness of the portion of the rechargeable implantable power source 131 positioned on the forehead may be extremely small, because this portion positioned on the forehead only comprises the photovoltaic module 11 and not a stack of the photovoltaic module 11 and the secondary battery 51. This is advantageous for the patient from the point of view of comfort and esthetics, the forehead being a particularly visible zone of the body; and
the area of the secondary battery 51 may be relatively large, especially larger than that of the photovoltaic module 11, because in humans the available area under the scalp is larger than the available area under the skin of the forehead. This makes it possible to use a secondary battery 51 of high energy capacity, and therefore to ensure the rechargeable implantable power source 131 has a long autonomy.

It will be noted that the thickness of the secondary battery may be relatively large, especially larger than that of the photovoltaic module, because the secondary battery is positioned in a zone generally covered with hair and therefore increasing its thickness does not inconvenience the patient from the esthetic point of view. Thus, the secondary battery may be replaced by a stack of secondary batteries. This makes it possible to increase the energy capacity of the energy storing subsystem and therefore to increase the autonomy of the rechargeable implantable power source.

The area of the rechargeable implantable power sources must be relatively large in order to ensure a satisfactory autonomy between two recharges. For example, for a thin-film secondary battery of typical energy capacity of 1 mW.h/cm², and an implantable medical device such as a cardiac stimulator consuming a typical average power of 30 μW, the area of the power source must be 30 cm² in order to ensure an autonomy of 1000 h, i.e. of about 40 days. However, objects of large area intended to be implanted under the skin are associated with a risk of causing poor vascularization of the skin, thereby possibly leading to necrosis.

The geometry of a rechargeable implantable power source may be adapted in order to surmount this problem of poor vascularization of the skin. More precisely, through-apertures extending right through the power source in its thickness direction may be arranged in order to allow blood vessels to pass through the power source.

FIG. 14 (top view) illustrates one way of arranging holes or apertures in a thin-film photovoltaic module of the energy harvesting subsystem. In the case illustrated in FIG. 14, the holes are arranged in the active zones of the photovoltaic module. FIG. 14 shows an active zone 141 of a photovoltaic module, of length denoted W_{PV} and width denoted w_{PV}. Holes, such as the hole 142, are arranged in the active zone 141. The holes pass right through the photovoltaic module in its thickness direction. The holes are rectangular in shape, of length denoted a and of width denoted b. The holes are regularly spaced, with a period denoted p in the length direction of the photovoltaic module; the period p (more generally the spacing between the holes or apertures, which may be variable) is generally comprised between 1 mm and 1 cm. Typically, the blood vessels irrigating human skin have a diameter of about a few tens or hundreds of microns, and a density of about a few vessels per mm² or a few tens of vessels per mm². Therefore, the length a and width b of a hole are advantageously comprised between 100 μm and 2 mm, i.e. the area of a hole is advantageously comprised between 0.01 mm² and 4 mm². In the typical case where W_{PV}=50 mm and where w_{PV}=8 mm, the area of the active zone 141 is equal to 400 mm². In this case, if the period p is for example 5 mm, then about ten holes are arranged in the active zone 141, this meaning that the apertured fraction of the active zone 141 is smaller than or equal to about 10%. Such a configuration makes it possible to limit the decrease in active zone area in the photovoltaic module (and therefore the decrease in electrical power generated under a given illumination), while providing a sufficient area for the blood vessels to pass through the photovoltaic module.

The arrangement of the holes in a thin-film photovoltaic module may be different to that illustrated in FIG. 14. For example, in the case illustrated in FIG. 15 (top view), the holes are arranged in the interconnect zones of the thin-film photovoltaic module. Relative to the configuration illustrated in FIG. 14, the configuration illustrated in FIG. 15 has the advantage of not decreasing the active area of the photovoltaic module. Thus, the effect of the holes on the electrical performance of the photovoltaic module is minimized. FIG. 15 shows a thin-film photovoltaic module 151 of rectangular shape, of length denoted L and width denoted I. The active zones, such as the zone 152, are rectangular in shape and of length denoted W_{PV} and width denoted w_{PV}. The interconnect zones, such as the zone 153, take the form of rectangular strips, of width denoted s_{PV}. Holes, such as the hole 154, are arranged in the interconnect zones of the photovoltaic module 151. The holes pass right through the photovoltaic module 151 in its thickness direction. The holes are rectangular in shape, of length denoted a and of width denoted b. The holes are regularly spaced, with a period denoted p in the length direction of the photovoltaic module 151. The length a and width b of a hole are advantageously comprised between 100 μm and 1 mm, i.e. the area of a hole is advantageously comprised between 0.01 mm² and 1 mm². In the typical case where L=50 mm and where s_{PV}=1 mm, the area of an interconnect zone (in the length direction of the photovoltaic module 151) is equal to 50 mm². In this case, if the period p is for example 5 mm, then about ten holes are arranged in a given interconnect zone, this meaning that the apertured fraction of a given interconnect zone is smaller than or equal to about 20%. Such a configuration makes it possible to obtain a good electrical interconnection between two adjacent active zones, while providing a sufficient area for the blood vessels to pass through the photovoltaic module 151.

FIG. 16 (top view) illustrates an arrangement of holes in a thin-film spiral coil of the energy harvesting subsystem. FIG. 16 shows a thin-film spiral coil 161 of rectangular shape, of length denoted L_{BOB} and width denoted I_{BOB}. The metal track 162 is spiral shaped. The width of the segments of the track 162 is denoted w_{BOB} and the spacing between the segments of the track 162 is denoted s_{BOB}. Holes, such as the hole 163, are arranged in the zones not covered by the metal track 162. Thus, the effect of the holes on the electrical performance of coil 161 is minimized. The holes are rectangular in shape, of length denoted a and width denoted b. The holes are regularly spaced, with a period p in the length and width directions of the spiral coil 161. The length a and the width b of a hole are advantageously comprised between 100 μm and 2 mm, i.e. the area of a hole is advantageously comprised between 0.01 mm² and 4 mm². The width b of a hole is advantageously smaller than the spacing s_{BOB}, and the period p is advantageously larger than the width w_{BOB}, so that the holes can be arranged in the zones located between two segments of the track 162.

FIG. 17 (top view) illustrates an arrangement of holes in a thin-film secondary battery of the energy storing subsystem. In the case illustrated in FIG. 17, the holes are arranged in the interconnect zones of the secondary battery. Thus the effect of the holes on the electrical performance of the secondary battery is minimized. FIG. 17 shows a thin-film secondary battery 171 that the same shape and size as the photovoltaic module 151, i.e. it is rectangular and of length L and width I. The active zones, such as the zone 172, are rectangular in shape, of length denoted W_{BAT} and of width denoted w_{BAT}. The interconnect zones, such as the zone 173, take the form of rectangular strips, of width denoted s_{BAT}. Holes, such as the hole 174, are arranged in the interconnect zones of the secondary battery 171. The holes pass right through the secondary battery in its thickness direction. The holes arranged in the secondary battery 171 are the same shape and size as the holes arranged in the photovoltaic module 151, i.e. they are rectangular and of length a and width b. The holes arranged in the secondary battery 171 are regularly spaced, with a period equal to that of the holes arranged in the photovoltaic module 151, i.e. a period p.

A rechargeable implantable power source 181 may comprise an assembly of the thin-film photovoltaic module 151 and the thin-film secondary battery 171, as illustrated in FIG. 18 (cross-sectional view). The photovoltaic module 151 is electrically connected to the secondary battery 171 in order to allow the secondary battery 171 to be recharged. The photovoltaic module 151 consists of a substrate 155 onto which a thin-film stack 156 has been deposited or transferred. Holes, such as the hole 154, pass right through the photovoltaic module 151 in its thickness direction. The secondary battery 171 consists of a substrate 175 onto which a thin-film stack 176 has been deposited or transferred. Holes, such as the hole 174, pass right through the secondary battery 171 in its thickness direction. The through-holes of the secondary battery 171 are the same shape and size and have the same spacing as the through-holes of the photovoltaic module 151. The through-holes of the photovoltaic module 151 and the through-holes of the secondary battery 171 are aligned, and thus contribute to forming through-holes that pass right through the rechargeable implantable power source 181 in its thickness direction. The assembly formed by the photovoltaic module 151 and the secondary battery 171 is encapsulated with a film stack 182. Advantageously, the film stack 182 also encapsulates the internal surfaces of the through-holes of the rechargeable implantable power source 181. This makes it possible to ensure that the rechargeable implantable power source 181 has a good resistance to moisture and oxygen. In order to form the film stack 182 on the internal surfaces of the holes, chemical vapor deposition and atomic layer deposition (ALD) processes are preferred, because they allow conformal thin films to be obtained.

The shape of the through-holes of the rechargeable implantable power source may be different from the shape shown in FIGS. 14, 15, 16, 17 and 18. For example, the holes may be circular in shape, this possibly making it easier to form them.

The holes are not necessarily regularly spaced, but may be positioned irregularly in the plane of the rechargeable implantable power source.

The internal surfaces of the holes may be covered with a gel promoting cellular growth, such as the gel “Matrigel”. The holes may also be filled with such a gel.

The implantable source may also comprise a data-processing subsystem, which may in turn comprise discrete or integrated electronic circuits such as microprocessors, microcontrollers or memories.

The implantable source may also comprise an energy management subsystem. The energy management subsystem may comprise discrete or integrated electronic circuits dedicated for example to DC/DC conversion (useful in the case where the energy harvesting subsystem delivers DC electrical power with too low or too high a voltage to efficiently recharge the energy storing subsystem) or to AC/DC conversion (useful in the case where the energy harvesting subsystem delivers AC electrical power that must be converted into DC electrical power in order to allow the energy storing subsystem to be recharged) or more generally to optimization of the recharging of the energy storing subsystem or to optimization of the power supply of the implantable medical device.

The implantable source may also comprise a communication subsystem. The communication subsystem may comprise components dedicated to the exchange of information with other communication systems located outside or inside the body of the patient. Information may be exchanged via light waves (preferably near-infrared light) or electromagnetic waves (preferably radio or microwave electromagnetic waves) or mechanical waves (preferably ultrasonic waves). Thus, the communication subsystem may comprise components such as light-emitting diodes (for exchanging information via light waves) or antennae (for exchanging information via electromagnetic waves) or piezoelectric transducers (for exchanging information via mechanical waves).

Most of the subsystems of the implantable device may take the form of ultra-thin objects. However, certain subsystems may be difficult to integrate in ultra-thin object form. This is for example the case of certain energy management subsystems employing relatively bulky electronic circuits and in particular discrete electronic components (capacitors and inductors). Therefore, in certain embodiments of the invention, the implantable device may comprise both ultra-thin zones, which will represent the majority of its total area, and substantially thicker zones, representing a minority of the total area. Advantageously, the maximum thickness of the ultra-thin zones is smaller than 1 mm (preferably smaller than 300 μm) and the maximum thickness of the thicker zones is smaller than 5 mm (preferably smaller than 1 mm). Advantageously, the ultra-thin zones represent more than 80% of the total area of the device, or at least of its power source. This solution makes it possible to preserve a maximum of patient comfort while benefiting from the functionalities of subsystems that are difficult to integrate in ultra-thin object form.

1. An implantable power source comprising at least one energy storing subsystem (171) produced in the form of a thin-film stack (175) on a substrate (176), characterized in that said energy storing subsystem has a plurality of through-apertures (174) in order to allow the development and passage of blood vessels.

2. The implantable power source as claimed in claim 1, in which each said aperture has an area comprised between 0.01 mm² and 4 mm².

3. The implantable power source as claimed in claim 1, in which the spacing between said apertures is comprised between 1 mm and 1 cm.

4. The implantable power source as claimed in claim 1, having a biocompatible coating (73, 182) covering at least one portion of its surface comprising the interior surface of said apertures.

5. The implantable power source as claimed in claim 4, in which said biocompatible coating comprises an exterior film (81) made of a biocompatible organic material and an interior film (82) made of an inorganic material that is impermeable to moisture and oxygen.

6. The implantable power source as claimed in claim 4, in which said biocompatible coating is substantially transparent at least in a spectral range in the visible or near infrared.

7. The implantable power source as claimed in claim 1, in which said apertures are completely or partially filled with a gel promoting cellular growth.

8. The implantable power source as claimed in claim 1, in which said energy storing subsystem has a plurality of active regions (172) separated by interconnect regions (173), at least certain of said apertures being produced in said active regions.

9. The implantable power source as claimed in claim 1, in which said energy storing subsystem has a plurality of active regions (172) separated by interconnect regions (173), at least certain of said apertures being produced in said interconnect regions.

10. The implantable power source as claimed in claim 1, also comprising at least one energy harvesting subsystem (151, 161) connected to said energy storing subsystem so as to allow the latter to be charged, said energy harvesting subsystem being in turn produced in the form of a thin-film stack (156) on a substrate (155) and having a plurality of said through-apertures (174).

11. The implantable power source as claimed in claim 10, in which said energy harvesting subsystem is chosen from a thin-film photovoltaic module (151) and a thin-film spiral coil (161).

12. The implantable power source as claimed in claim 10, in which said energy harvesting subsystem has at least one active region (152, 162) and at least one inactive or interconnect region (153), at least certain of said apertures being produced in said active region(s).

13. The implantable power source as claimed in claim 10, in which said energy harvesting subsystem has at least one active region (152, 162) and at least one inactive or interconnect region (153), at least certain of said apertures being produced in said inactive or interconnect region(s).

14. The implantable power source as claimed in claim 10, in which said energy storing subsystem and said energy harvesting subsystem comprise thin-film stacks deposited on or transferred to respective substrates (12, 52) and are in turn stacked.

15. The implantable power source as claimed in claim 10, in which said energy storing subsystem and said energy harvesting subsystem are stacked on a common substrate (122).

16. The implantable power source as claimed in claim 10, in which said energy storing subsystem and said energy harvesting subsystem comprise thin-film stacks deposited on or transferred to two opposite sides of a common substrate (12).

17. The implantable power source as claimed in claim 1, in which said or each said substrate is flexible or shapeable.

18. The implantable power source as claimed in claim 10, in which said energy storing subsystem and said energy harvesting subsystem are arranged side-by-side.

19. The implantable power source as claimed in claim 1, having, over at least 80% of its area, a thickness smaller than or equal to 1 mm.

20. An implantable device comprising an implantable power source as claimed in claim 1 and a medical apparatus connected to said energy storing subsystem in order to be powered.
